(19) United States
(12) Patent Application Publication
(10) Pub. No.: US 2023/0081265 A1
(43) Pub. Date: Mar. 16, 2023

(54) ANTIBODY FORMULATION

(71) Applicants: The Board of Trustees of the Leland Stanford Junior University, Stanford, CA (US); Forty Seven, Inc., Foster City, CA (US)

(72) Inventors: Ravindra Majeti, Palo Alto, CA (US); Irving L. Weissman, Stanford, CA (US); Phuong Nguyen, Redwood City, CA (US)

(21) Appl. No.: 17/916,994

(22) PCT Filed: Mar. 30, 2021
(86) PCT No.: PCT/US21/24937

§ 371 Date: Oct. 4, 2022

Related U.S. Application Data
(60) Provisional application No. 63/005,755, filed on Apr. 6, 2020.

(51) Int. Cl. C07K 16/28; A61K 47/26; A61K 9/08

(52) U.S. Cl. CPC C07K 16/2896; A61K 47/26; A61K 9/08; C07K 2317/565; C07K 2317/24; C07K 2317/94

(57) ABSTRACT

Formulations of anti-CD47 antibodies having a pharmacologically acceptable concentration and stable shelf life are provided.

### CROSS-REFERENCE TO RELATED APPLICATIONS

The present application claims the benefit of and priority to U.S. Provisional Patent Application No. 63/005,755, filed Apr. 6, 2020, the entire disclosure of which is hereby incorporated by reference herein in its entireties for all purposes.

### BACKGROUND

Immunoglobulin proteins are multifunctional components of the immune system that facilitate cellular and humoral reactions to a variety of antigens. One form of immunotherapy exploits the capabilities of the innate immune system. The cell surface protein CD47 through engagement with the phagocyte receptor, SIRPa, provides a key “don't eat-me” signal that can turn off phagocytosis of cells that express CD47. Blocking CD47 mediated engagement of SIRPa on a phagocyte can enhance phagocytic removal of a targeted cell. Anti-CD47 antibody treatment has also been shown to enable macrophage phagocytosis of cancer cells.

Providing stable, therapeutic formulations of anti-CD47 antibodies is a critical step in developing a clinically relevant therapy. However, CD47 is a widely expressed antibody, found at some level on most human cells. Binding anti-CD47 antibody to targets on cell surfaces may trigger internalization of the complex into the cells followed by subsequent lysosomal degradation of the complex. Typically, clearance of mAb that binds to membrane antigen is faster at low doses as the unbound targets will “sop up” antibody, serving as a sink (this phenomenon is referred to as the “antigen sink”). The result is that relatively high doses of the antibody may be required.

Further, because antibodies are large and complex molecules, their formulation poses special problems. For a protein to remain biologically active, a formulation must preserve intact the conformational integrity of at least a core sequence of the protein's amino acids while at the same time protecting the protein's multiple functional groups from degradation. Degradation pathways for proteins can involve chemical instability, for example from deamidation, racemization, hydrolysis, oxidation, beta elimination or disulfide exchange; or physical instability from denaturation, aggregation, precipitation or adsorption.

The process of formulating an antibody therapeutic requires first an understanding of how the protein handles exposure to stressors that may be encountered during manufacturing or storage such as freeze/thaw, agitation/shear, thermal stability. Antibodies have a propensity to aggregate at high concentrations making formulation optimization difficult, and there is a relatively short list of buffers and excipients that are currently FDA approved for formulation of antibodies, which may limit the space for high concentration optimization.

Specific formulations providing for stable storage of antibodies for clinical use remains a challenge.

Related publications include U.S. Pat. Nos. 8,562,997; 9,399,682; 9,017,675; 9,382,320; 9,151,760; 8,758,750; 8,361,736; 8,709,429; 9,193,955; and 7,514,229 and International Patent Applications US2016/049016; US2016/030997; US2016/036520; US2015/046976; US2015/044304; US2015/057233; US2015/026491; US2015/019954; US2015/010650; US2014/035167; US2014/018743; US2014/038485; US2013/021937; and US2011/066580, each herein specifically incorporated by reference.

### SUMMARY

Formulations of anti-CD47 antibodies are provided, and in particular aqueous and lyophilized formulations of anti-CD47 antibodies having a pharmacologically acceptable concentration and stable shelf life.

In some embodiments, the anti-CD47 antibody in the formulation is not subject to prior lyophilization, e.g. is in a liquid formulation. In some embodiments, the anti-CD47 antibody in the formulation is a monoclonal antibody. In some embodiments, the anti-CD47 antibody in the formulation is a full-length antibody. In some embodiments, the anti-CD47 antibody in the formulation is an IgG antibody. In some embodiments, the anti-CD47 antibody in the formulation is a humanized antibody. In some embodiments the antibody comprises a human IgG4 constant chain. In some embodiments the antibody is magrolimab.

In some embodiments, the anti-CD47 antibody comprises an HCDR1 region comprising of sequence (SEQ ID NO: 1), an HCDR2 region comprising of sequence (SEQ ID NO: 2), an HCDR3 region comprising of sequence (SEQ ID NO: 3), an LCDR1 region comprising of sequence (SEQ ID NO: 4), an LCDR2 region comprising of sequence (SEQ ID NO: 5), and an LCDR3 region comprising of sequence (SEQ ID NO: 6). In some embodiments such an antibody comprises a human IgG4 constant region sequence.

In some embodiments the formulation is a liquid formulation comprising: 10-160 mg/ml, e.g., 10-100 mg/ml, e.g., 10-80 mg/ml of an anti-CD47 antibody; a pharmaceutically acceptable buffer at a concentration of 5-20 mM; a stabilizer, and a surfactant, wherein the formulation has a pH of from about pH 4-6.5. In some embodiments the anti-CD47 antibody is magrolimab.

In some embodiments the formulation is a liquid formulation comprising 10-20 mg/ml magrolimab; 10 mM acetate buffer; 5% w/v sorbitol; 0.01%-0.04% polysorbate 20, e.g., 0.01% -0.02% polysorbate 20; the formulation at a pH of from 4.5-5.5.

In some embodiments the formulation is a liquid formulation comprising 10-20 mg/ml magrolimab; 10 mM acetate buffer; 2-12% w/v sucrose, e.g., 9% w/v sucrose; 0.01%-0.04% polysorbate 20, e.g., 0.01%-0.02% polysorbate 20; the formulation at a pH of from 4.5 — 5.5.

In some embodiments the formulation is a liquid formulation comprising 10-20 mg/ml magrolimab; 10 mM acetate buffer; 2-12% w/v trehalose, e.g., 9% w/v trehalose; 0.01%-0.04% polysorbate 20, e.g., 0.01%-0.02% polysorbate 20; the formulation at a pH of from 4.5-5.5.

In some embodiments the formulation provides for stability of the antibody in storage, e.g. maintaining at least 50% activity, at least 75% activity, at least 95% activity after storage at 2-8° C. for a period of greater than 8 weeks, greater than 16 weeks, greater than 24 weeks, greater than 48 weeks, greater than 12 months, greater than 2 years, greater than 3 years, greater than 4 years, greater than 5 years. Stability after 12 months, 2 years, 3 years, 4 years or 5 years may be 100±50%.

In some embodiments a method of use is provided, comprising administering to a patient in need thereof an effective dose of an antibody formulation comprising 10-80 mg/ml, e.g., 20-50 mg/ml magrolimab; 10 mM acetate buffer; 5% w/v sorbitol; 0.01%-0.04% polysorbate 20, e.g., 0.01%-0.02% polysorbate 20; the formulation at a pH of from 4.0-5.5, e.g., 4.0-5.3, e.g., 4.0-5.0.

In some embodiments a method of use is provided, comprising administering to a patient in need thereof an effective dose of an antibody formulation comprising 10-80 mg/ml, e.g., 20-50 mg/ml magrolimab; 10 mM acetate buffer; 9% w/v sucrose; 0.01%-0.04% polysorbate 20, e.g., 0.01%-0.02% polysorbate 20; the formulation at a pH of from 4.0-5.5, e.g., 4.0-5.3, e.g., 4.0-5.0.

In some embodiments a method of use is provided, comprising administering to a patient in need thereof an effective dose of an antibody formulation comprising 10-80 mg/ml, e.g., 20-50 mg/ml magrolimab; 10 mM acetate buffer; 9% w/v trehalose; 0.01%-0.04% polysorbate 20, e.g., 0.01%-0.02% polysorbate 20, at a pH of from 4.0-5.5, e.g., 4.0-5.3, e.g., 4.0-5.0.

### BRIEF DESCRIPTION OF THE DRAWINGS

FIG. 1. Surface tension of magrolimab formulation as a function of polysorbate 20 added.

FIGS. 2A-2D. Main Peak WCX-HPLC Results

FIGS. 3A-3D. Pre-Peak WCX-HPLC Results

FIGS. 4A-4D. SE-HPLC Monomer Results

FIGS. 5A-5D. SE-HPLC Aggregates/Pre-Peak Results

FIG. 6. Viscosity of magrolimab measured as a function of concentration

FIG. 7. Various stabilizing excipients studied with magrolimab

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

By “comprising” it is meant that the recited elements are required in the composition/method/kit, but other elements may be included to form the composition/method/kit etc. within the scope of the claim. For example, a composition may comprise agents that promote the stability, agents that promote solubility, adjuvants, etc. as will be readily understood in the art, with the exception of elements that are encompassed by any negative provisos.

By “consisting essentially of”, it is meant a limitation of the scope of composition or method described to the specified materials or steps that do not materially affect the basic and novel characteristic(s) of the subject invention. For example, an antibody “consisting essentially of” a disclosed sequence has the amino acid sequence of the disclosed sequence plus or minus about 5 amino acid residues at the boundaries of the sequence based upon the sequence from which it was derived, e.g. about 5 residues, 4 residues, 3 residues, 2 residues or about 1 residue less than the recited bounding amino acid residue, or about 1 residue, 2 residues, 3 residues, 4 residues, or 5 residues more than the recited bounding amino acid residue.

By “consisting of”, it is meant the exclusion from the composition, method, or kit of any element, step, or ingredient not specified in the claim.

General methods in molecular and cellular biochemistry can be found in such standard textbooks as Molecular Cloning: A Laboratory Manual, 3rd Ed. (Sambrook et al., CSH Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

As used herein, “antibody” includes reference to an immunoglobulin molecule immunologically reactive with a particular antigen, and includes both polyclonal and monoclonal antibodies, e.g. an entire tetrameric IgG protein. The term also includes genetically engineered forms such as chimeric antibodies (e.g., humanized murine antibodies) and heteroconjugate antibodies. The term “antibody” also includes antigen binding forms of antibodies, including fragments with antigen-binding capability (e.g., Fab′, F(ab′)₂, Fab, Fv and rlgG. The term also refers to recombinant single chain Fv fragments (scFv). The term antibody also includes bivalent or bispecific molecules, diabodies, triabodies, and tetrabodies.

Antibodies also exist as a number of well-characterized fragments produced by digestion with various peptidases. Thus, pepsin digests an antibody below the disulfide linkages in the hinge region to produce F(ab)′₂, a dimer of Fab which itself is a light chain joined to VH-CH, by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin, particularly in IgG4 Fc region.

The “CDRs” herein can be defined by Chothia et al; Kabat et al.; etc. See Chothia C, Lesk A M. (1987) Canonical structures for the hypervariable regions of immunoglobulins. J Mol Biol., 196(4):901-17, which is incorporated by reference in its entirety. See Kabat E. A, Wu T. T., Perry H. M., Gottesman K. S. and Foeller C. (1991). Sequences of Proteins of Immunological Interest. 5th edit., NIH Publication no. 91-3242, US Dept. of Health and Human Services, Washington, D.C., which is incorporated by reference in its entirety.

The term “epitope” includes any protein determinant capable of specific binding to an antibody or otherwise interacting with a molecule. Epitopic determinants generally consist of chemically active surface groupings of molecules such as amino acids or carbohydrate or sugar side chains and can have specific three-dimensional structural characteristics, as well as specific charge characteristics. An epitope may be “linear” or “conformational”. The term “linear epitope” refers to an epitope with all of the points of interaction between the protein and the interacting molecule (such as an antibody) occur linearly along the primary amino acid sequence of the protein (continuous). The term “conformational epitope” refers to an epitope in which discontinuous amino acids that come together in three-dimensional conformation. In a conformational epitope, the points of interaction occur across amino acid residues on the protein that are separated from one another.

“Binds the same epitope as” means the ability of an antibody or other binding agent to bind to CD47 and having the same epitope as the exemplified antibody. The epitopes of the exemplified antibody and other antibodies to CD47 can be determined using standard epitope mapping techniques. Epitope mapping techniques, well known in the art include Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al, (1984) Proc. Natl. Acad. Sci. USA 8:3998-4002; Geysen et al, (1985) Proc. Natl. Acad. Sci. USA 82:78-182; Geysen et al, (1986) Mol. Immunol. 23:709-715. Similarly, conformational epitopes are readily identified by determining spatial conformation of amino acids such as by, e.g., hydrogen/deuterium exchange, x-ray crystallography and two-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al, (1981)

Proc. Natl. Acad. Sci USA 78:3824-3828; for determining antigenicity profiles, and the Kyte-Doolittle technique, Kyte et al, (1982) J. Mol. Biol. 157: 105-132; for hydropathy plots.

### Formulations

Formulations of the present disclosure comprise 10-160 mg/ml, e.g., 10-100 mg/ml, e.g., 10-80 mg/ml of an anti-CD47 antibody; and pharmaceutically acceptable excipients comprising buffer at a concentration of 5-20 mM; a stabilizer, and a surfactant, wherein the formulation has a pH of from about pH 4-6.5.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. Various pharmaceutically acceptable diluents, carriers, and excipients, and techniques for the preparation and use of pharmaceutical compositions will be known to those of skill in the art in light of the present disclosure. Illustrative pharmaceutical compositions and pharmaceutically acceptable diluents, carriers, and excipients are also described, e.g., in Remington: The Science and Practice of Pharmacy 20th Ed. (Lippincott, Williams & Wilkins 2003); Loyd V. Allen Jr (Editor), “Remington: The Science and Practice of Pharmacy,” 22nd Edition, 2012, Pharmaceutical Press; Brunton, Knollman and Hilal-Dandan, “Goodman and Gilman's The Pharmacological Basis of Therapeutics,” 13th Edition, 2017, McGraw-Hill Education/Medical; McNally and Hastedt (Editors), “Protein Formulation and Delivery, 2nd Edition, 2007, CRC Press; Banga, “Therapeutic Peptides and Proteins: Formulation, Processing, and Delivery Systems,” 3rd Edition, 2015, CRC Press; Lars Hovgaard, Frokjaer and van de Weert (Editors), “Pharmaceutical Formulation Development of Peptides and Proteins,” 2nd Edition, 2012, CRC Press; Carpenter and Manning (Editors), “Rational Design of Stable Protein Formulations: Theory and Practice,” 2002, Springer (Pharmaceutical Biotechnology (Book 13)); Meyer (Editor), “Therapeutic Protein Drug Products: Practical Approaches to Formulation in the Laboratory, Manufacturing, and the Clinic, 2012, Woodhead Publishing; and Shire, “Monoclonal Antibodies: Meeting the Challenges in Manufacturing, Formulation, Delivery and Stability of Final Drug Product, 2015, Woodhead Publishing.

Pharmaceutical compositions incorporating an antibody, as described herein, for parenteral administration are formulated in a unit dosage injectable form (e.g., solution, suspension, emulsion) in association with a pharmaceutically acceptable, parenteral vehicle and can be sterile and substantially isotonic (250-350 mOsm/kg water), and manufactured under GMP conditions. Pharmaceutical compositions can be provided in unit dose form (i.e., the dose for a single administration). Pharmaceutical compositions can be formulated using one or more pharmaceutically acceptable carriers, diluents, excipients or auxiliaries. The formulation depends on the route of administration chosen. For injection, antibodies can be formulated in aqueous solutions, e.g., in physiologically compatible buffers such as phosphate-buffered saline, amino acid buffers (e.g., charged amino acids, including without limitation, histidine, aspartate, glutamate, asparagine, glutamine, lysine, arginine), a Tris buffer, Hank's solution, Ringer's solution, dextrose solution, and 5% human serum albumin, or acetate buffer (to reduce discomfort at the site of injection), and the additional buffers listed herein. The solution can contain formulatory agents such as suspending, stabilizing and/or dispersing agents. Alternatively, antibodies can be in lyophilized form for constitution with a suitable vehicle, e.g., sterile pyrogen-free water, before use. The formulation of and delivery methods of pharmaceutical compositions will generally be adapted according to the site and the disease to be treated. Exemplary formulations include, but are not limited to, those suitable for parenteral administration, e.g., intravenous, or subcutaneous administration.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomersAs used herein, “buffer” refers to a buffered solution that resists changes in pH by the action of its acid-base conjugate components. Buffers useful in pharmaceutical formulations, particularly formulations for parenteral administration, may be used in the present formulation, and include without limitation: acetate buffers, e.g. acetic acid/sodium acetate; histidine; phosphate, citrate, lactate; aspartate; succinate; malate; fumarate; gluconate; glutamate. In some embodiments the buffer is an acetate buffer. In some embodiments the buffer is a histidine buffer.

In some embodiments, the buffer in the formulation is in a concentration of 5 to 50 mM. In some embodiments, the buffer in the formulation is in a concentration of 7.5 to 25 mM. In some embodiments, the buffer in the formulation is in a concentration of 10 to 20 mM. In some embodiments, the buffer in the formulation is acetate buffer or histidine buffer.

In an aqueous formulation, the concentration of buffer may be about 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM. In certain embodiments the concentration is from 8 to 12 mM, e.g. 10 mM. Such aqueous formulations can be lyophilized, as appropriate or desired.

The pH of the buffered formulation may be from pH 4 to pH 6.5. In some embodiments the pH is from pH 4.5 to 6. In some embodiments the pH is from pH 4.7 to 5.3. In some embodiments the pH is from pH 4.75 to pH 5.25. In some embodiments the pH is from pH 4.9 to pH 5.1.

In some embodiments the pH of the formulation is about pH 4.5, pH 4.6, pH 4.7, pH 4.8, pH 4.9, pH 5, pH 5.1, pH 5.2, pH 5.3, pH 5.4, pH 5.5. In certain embodiments the pH is 4.9 to 5.1, e.g. pH 5.

Stabilizers, cryoprotectants, and/or lyoprotectants are included to improve the stability of the antibody across conditions in storage, transfer and use. Suitable stabilizers include without limitation, carbohydrates, amino acids, cosolvents, antioxidants, chelators, salts, tonicity modifiers, ionic strength modifiers.

The formulation can contain any desired free amino acid, which can be in the L-form, the D-form or any desired mixture of these forms. In one aspect, free amino acids that can be included in the formulation include, for example, histidine, alanine, arginine, glycine, glutamic acid, serine, lysine, tryptophan, valine, cysteine, methionine, and combinations thereof. Some amino acds can stabilize the proteins against degradation during manufacturing, drying, lyophilization and/or storage, e.g., through hydrogen bonds, salt bridges, antioxidant properties, or hydrophobic interactions or by exclusion from the protein surface. Amino acids can act as tonicity modifiers or can act to decrease viscosity of the formulation. In another aspect, free amino acids.,such as histidine, glycine and arginine, can act as lyoprotectants, and do not crystallize when lyophilized as components of the formulation. Free amino acids, such as glutamic acid and histidine, alone or in combination, can act as buffering agents in aqueous solution in the pH range of 5 to 7.5.

A “saccharide” herein is a compound that has a general formula (CH₂O) and derivatives thereof, including monosaccharides, disaccharides, trisaccharides, polysaccharides, sugar alcohols. reducing sugars, nonreducing sugars, and the like. Examples of saccharides herein include glucose, sucrose, trehalose, lactose, fructose, maltose, dextran, erythritol, glycerol, arabitosylitosorbitol, mannitol, melibiose, melezitose, raffmose, mannotriose, stachyose, maltose, lacrulose, maltulose, glucitol, maltitol, lactitol, iso-maltulose, sorbitol, and the like. A saccharide can be a lyoprotectant. In one aspect, a saccharide that does not crystallize is a lyoprotectant, such as sucrose or trehalose. In one aspect, a saccharide herein is a nonreducing disaccharide, such as sucrose, trehalose or sorbitol.

The term “chelator” refers to an agent that binds to an atom through more than one bond. In one aspect, examples of chelators herein include citrate, EDTA, EGTA, dimercaprol, diethylenetriaminepentaacetic acid, and N,N-bis(carboxymethyl)glycine. In another aspect the chelator is citrate or EDTA.

The term antioxidant refers to an agent that inhibits the oxidation of other molecules. Examples of antioxidants herein include citrate, lipoic acid, uric acid, glutathione, tocopherol, carotene, lycopene, cysteine, phosphonate compounds, e.g. etidronic acid, desferoxamine and malate.

Specific stabilizers of interest for the formulations of the disclosure may include one or more of sorbitol, e.g. at a concentration of from about 2% to about 12%, e.g., at a concentration from about 2.5% to about 10%; sucrose, e.g. at a concentration of from about at a concentration of from about 2% to about 12%, e.g., at a concentration from about 2.5% to about 10%; trehalose , e.g. at a concentration of from about at a concentration of from about 2% to about 12%, e.g., at a concentration from about 2.5% to about 10%; NaCl at a concentration of from about 100-200 mM; arginine at a concentration of from about 15 mM to 75 mM; or a combination thereof, e.g. arginine and sorbitol, arginine and sucrose, arginine and trehalose, sucrose and sorbitol, and the like.

In some embodiments the stabilizer is sorbitol at a concentration of about 2.5% w/v, 3%, 4%, 5%, 6%, 7%, 8%, 9%, 10%. In certain embodiments the stabilizer is sorbitol at a concentration of about 5% weight/volume in an aqueous buffer.

A “surfactant” refers to a surface-active agent. Surfactants are included in the formulation, primarily to reduce interaction of protein at solid/liquid interfaces, liquid/air interfaces, and protein/protein interfaces. It reduces the formation of particles by reducing adsorption of protein to surfaces, as well as aggregation due to stresses from agitation, free/thaw, and shear. Additionally, it may enhance solubility of the protein. Generally categories of surfactants are anionic, e.g. sodium linear alkylbenzene sulphonate (LABS); sodium lauryl sulphate; sodium lauryl ether sulphates; petroleum sulphonates; linosulphonates; naphthalene sulphonates, branched alkylbenzene sulphonates; linear alkylbenzene sulphonates; alcohol sulphates; cationic, e.g. Stearalkonium chloride; benzalkonium chloride; quaternary ammonium compounds; amine compounds, non-ionic, e.g. dodecyl dimethylamine oxide; coco diethanol-amide alcohol ethoxylates; linear primary alcohol polyethoxylate; alkylphenol ethoxylates; alcohol ethoxylates; EO/PO polyol block polymers; polyethylene glycol esters; fatty acid alkanolamides; amphoteric: Cocoamphocarboxyglycinate; cocamidopropylbetaine; betaines; imidazolines.

In addition to those listed above, suitable nonionic surfactants include alkanolamides, amine oxides, block polymers, ethoxylated primary and secondary alcohols, ethoxylated alkylphenols, ethoxylated fatty esters, sorbitan derivatives, glycerol esters, propoxylated and ethoxylated fatty acids, alcohols, and alkyl phenols, alkyl glucoside glycol esters, polymeric polysaccharides, sulfates and sulfonates of ethoxylated alkylphenols, and polymeric surfactants. Suitable anionic surfactants include ethoxylated amines and/or amides, sulfosuccinates and derivatives, sulfates of ethoxylated alcohols, sulfates of alcohols, sulfonates and sulfonic acid derivatives, phosphate esters, and polymeric surfactants

In some embodiments the surfactant of the formulation is polysorbate 20, polysorbate 40, polysorbate 60, polysorbate 80, or pluronic F68. In certain embodiments the surfactant is polysorbate 20.

The ideal concentration of surfactant is chosen to be at least the concentration that fully saturates the hydrophobic surfaces of the protein, although a non-fully saturating concentration can also provide benefits. For example, surface tensiometry may be used to evaluate the interaction of surfactant with antibody to determine the concentration at which the surfactant is fully adsorbed to the surface of the protein. The amount of surfactant will increase with an increase in protein concentration, due to the increase in the surface area of protein in the formulation. Therefore, the concentration of surfactant may be increased with an increase in protein concentration.

In some embodiments the concentration of surfactant is from about 0.002% to about 0.02% in a liquid formulation with 20-25 mg/ml antibody; and may be from about 0.0075% to about 0.015%. from about 0.008% to about 0.012%. In some embodiments the surfactant concentration is about 0.004%, 0.006%, 0.01%, 0.011%, 0.012%, 0.013%, 0.014%, 0.015%.

In some embodiments the surfactant concentration is at a surfactant:protein molar range of from about 0.2 to about 2, and may be from about 0.2, about 0.3, about 0.4, about 0.5, about 0.6, up to about 2, up to about 1.5, up to about 1.2. The surfactant in such embodiments may be, for example, a polysorbate.

A “stable” formulation is one that can be administered to patients after storage. In aspects, the formulation essentially retains its physical and chemical properties, as well as its biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example.

Stability can be evaluated qualitatively and/or quantitatively in a variety of different ways, including evaluation of soluble aggregate formation (for example using size exclusion chromatography, by measuring turbidity) and visible particle formation by by visual inspection); by assessing charge heterogeneity using ion exchange chromatography (IEC) or imaged capillary isoelectric focusing (icIEF), size exclusion chromatography (SE-HPLC), CE-SDS or SDS-PAGE analysis to compare reduced and intact antibody, sub-visible particle formation by light obscuration, microflow imaging, or microscopy; evaluating biological activity or antigen binding function of the antibody; etc. Instability may involve any one or more of: aggregation, deamidation (e.g. Asn deamidation), oxidation (e.g. Met oxidation), isomerization (e.g. Asp isomerization), clipping/hydrolysis/fragmentation (e.g. hinge region fragmentation), succinimide formation, unpaired cysteine(s), N-terminal extension, C-terminal processing, glycosylation differences, etc.

As used herein, “biological activity” of a monoclonal antibody refers to the ability of the antibody to bind to antigen. It can further include antibody binding to antigen and resulting in a measurable biological response which can be measured in vitro or in vivo.

In some embodiments the stable pharmaceutical formulation is stable at a temperature of from about 2° C. to about 8° C. for at least about 1, 2, 3, 4, 5, 6, 7, 8, 9, 10, 11, 12, 13, 14, 15, 16 or more weeks, and up to 3 months, 6 months, 9 months, 12 months or more. In certain embodiments, the stable pharmaceutical formulation is stable at a temperature of from about 2° C. to about 8° C. for at least about 1, 2, 4, 6, 8, 12 or more months. In certain embodiments, the stable pharmaceutical formulation is stable at a temperature of from about 2° C. to about 8° C. for at least 1, 2, 3, 4, 5, 6, 7, 8, 9, 10, 11, 12 or more months.

In a further embodiment the anti-CD47 antibody in the formulation retains at least 50%, at least 70%, at least 80%, at least 90%, at least 95%, at least 97%, at least 99% of its biological activity after storage. In some embodiment the biological activity is measured by antibody binding to CD47. In some embodiment the biological activity is measured by antibody binding to CD47 in a FACS CD47 binding assay. In some embodiment the biological activity is measured by ADCP activity of said anti-CD47 antibody.

In another aspect, provided herein is an article of manufacture comprising a container holding the stable pharmaceutical formulation as disclosed herein. In an embodiment said container is a glass vial, e.g. a type 1 glass vial, polymeric vial (e.g., cyclic olefin polymer, cyclic olefin copolymer, high-density polyethylene (HDPE), polycarbonate, polyethylene terephthalate glycol (PETG)), polymeric bag or pouch (e.g., low-density polyethylene (LDPE), ethyl vinyl acetate (EVA)), or a metal alloy container.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

A dosage unit of the present formulation may be 1 ml., 2 ml., 5 ml., 10 ml., 15 ml, 20 ml, 30 ml, 40 ml, 50 ml, 60m1 at a concentration of from about 10 to about 160 mg/ml, at a concentration of from about 10 to about 100 mg/ml, at a concentration of from about 10 to about 80 mg/ml, at a concentration of from about 15 to about 50 mg/ml, at a concentration of from about 10 to about 25 mg/ml, at a concentration of from about 15 to about 20 mg/ml; and may be at a concentration of about 20 mg/ml. In some embodiments a dosage unit is 10 ml. In some embodiments a dosage unit is 20 ml. In some embodiments a dosage unit is 30 ml. In some embodiments a dosage unit is 40 ml. In some embodiments a dosage unit is 50 ml. In some embodiments a dosage unit is 60 ml.

A “therapeutically effective dose” or “therapeutic dose” is an amount sufficient to effect desired clinical results (i.e., achieve therapeutic efficacy), and may utilize 1, 2, 3, 4, 5, 6, 7, 8, 9, 10, 11, 12, 13, 14, 15, or more dosage units of the formulation. For purposes of this invention, a therapeutically effective dose of an anti-CD47 agent is an amount that is sufficient to palliate, ameliorate, stabilize, reverse, prevent, slow or delay the progression of the disease state by increasing phagocytosis of a target cell (e.g., a target cell); for example to reduce the number of tumor cells in the blood, bone marrow, etc. Thus, a therapeutically effective dose of an anti-CD47 agent reduces the binding of CD47 on a target cell, to SIRPa on a phagocytic cell, at an effective dose for increasing the phagocytosis of the target cell.

In some embodiments, the anti-CD47 antibody in the formulation is a monoclonal antibody. In some embodiments, the anti-CD47 antibody in the formulation is a full-length antibody. In some embodiments, the anti-CD47 antibody in the formulation is an IgG antibody. In some embodiments, the anti-CD47 antibody in the formulation is a humanized antibody. In some embodiments the antibody comprises a human IgG4 constant chain.

In some embodiments, the anti-CD47 antibody comprises an HCDR1 region comprising of sequence (SEQ ID NO: 1), an HCDR2 region comprising of sequence (SEQ ID NO: 2), an HCDR3 region comprising of sequence (SEQ ID NO: 3), an LCDR1 region comprising of sequence (SEQ ID NO: 4), an LCDR2 region comprising of sequence (SEQ ID NO: 5), and an LCDR3 region comprising of sequence (SEQ ID NO: 6). In some embodiments such an antibody comprises a human IgG4 constant region sequence. SEQ ID NO:7, 8 and 9 provide exemplary heavy chain variable region sequences; and SEQ ID NO:10, 11, 12 provide exemplary light chain variable region sequences.

In some embodiments the antibody is magrolimab, and comprises the heavy chain variable region sequence of SEQ ID NO:8 and the light chain variable region sequence of SEQ ID NO:11, with an IgG4 heavy chain constant region. Optionally the IgG4 constant region has an amino acid modification to stabilize the hinge region, e.g. an S228P amino acid substitution.

### Methods of Use

The compositions can be administered for therapeutic treatment. Compositions are administered to a patient in an amount sufficient to substantially enhance phagocytosis of targeted cells. An amount adequate to accomplish this is defined as a “therapeutically effective dose.”, which may provide for an improvement in overall survival rates. Single or multiple administrations of the compositions may be administered depending on the dosage and frequency as required and tolerated by the patient. The particular dose required for a treatment will depend upon the medical condition and history of the mammal, as well as other factors such as age, weight, gender, administration route, efficiency, etc.

The formulations described herein find use in, for example in treating or reducing cancer, reducing infection in a regimen comprising contacting the targeted cells with an effective dose of the formulation that blockades CD47 activity; etc. Effective doses of the agent of the present invention for the treatment of cancer vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

A “patient” includes both humans and other animals, particularly mammals, including pet and laboratory animals, e.g. mice, rats, rabbits, etc. Thus, the methods are applicable to both human therapy and veterinary applications. In one embodiment the patient is a mammal, preferably a primate. In other embodiments the patient is human.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for detection, analysis, or treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable number of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

The term “diagnosis” is used herein to refer to the identification of a molecular or pathological state, disease or condition, such as the identification of a molecular subtype of breast cancer, prostate cancer, or other type of cancer.

The term “prognosis” is used herein to refer to the prediction of the likelihood of cancer-attributable death or progression, including recurrence, metastatic spread, and drug resistance, of a neoplastic disease, such as ovarian cancer. The term “prediction” is used herein to refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence.

In an embodiment the cancer is a hematologic cancer or myelodysplastic syndrome, e.g. non-Hodgkin's lymphoma, chronic lymphocytic leukemia, acute lymphoblastic leukemia, acute myelogenous leukemia, myelodysplastic syndrome; multiple myeloma, and the like. In embodiments, the non-Hodgkin's lymphoma is selected from the group consisting of follicular lymphoma, small lymphocytic lymphoma, mucosa-associated lymphoid tissue, marginal zone, diffuse large B cell, Burkitt's, and mantle cell. In some embodiments MDS is treated. In some embodiments AML is treated. In other embodiments the cancer is a carcinoma, sarcoma, myeloma, glioma, etc.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease, and/or symptoms of the disease. “Treatment,” as used herein, may include treatment of a tumor in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

Treating may refer to any indicia of success in the treatment or amelioration or prevention of an cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with cancer or other diseases. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

In some embodiments, the therapeutic dosage of the anti-CD47 antibody formulation may range from about 1 to 100 mg/kg, e.g., from about 10 to 90 mg/kg, e.g., from about 10 to 60 mg/kg, and more usually 10 to 50 mg/kg, of the host body weight. For example, dosages can be about 20, 30, 40, 45, 50, 60 mg/kg body weight or within the range of 20-60, 20-50 or 30-60 mg/kg. An exemplary treatment regime entails administration once every 3 days, every week, every two weeks, every three weeks, or once a month, once every 6 weeks, or once every 3 to 6 months. Therapeutic entities of the present invention are usually administered on multiple occasions. Intervals between single dosages can be daily, weekly, monthly or yearly. Intervals can also be irregular as indicated by measuring blood levels of the therapeutic entity in the patient. Dosage and frequency vary depending on the half-life of the polypeptide in the patient.

Toxicity can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

Also within the scope of the invention are kits comprising the formulations of the invention and instructions for use. The kit can further contain an aid for liquid extraction; a least one additional reagent, e.g. a chemotherapeutic drug, ESA, etc. Kits typically include a label indicating the intended use of the contents of the kit. The term label includes any writing, or recorded material supplied on or with the kit, or which otherwise accompanies the kit.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Formulations tested for stability of the humanized 5F9-G4 antibody (magrolimab).

The stability of Hu5F9-g4 in various formulation conditions including pH, tonicity modifier and surfactant was initially examined. This study was performed with weak cation exchange HPLC (WCX-HPLC), size-exclusion HPLC (SE-HPLC) and sodium dodecyl sulfate polyacrylamide gel electrophoresis (SDS-PAGE) analyses, which are effective stability-indicating assays for the product.

The product turns hazy when agitation stress is introduced. Therefore, polysorbate 20 as a surfactant was added to the Hu5F5-g4 formulations to stabilize the product during stress. Additional acute stress examinations revealed that the product was not susceptible to consecutive freeze/thaw cycles, agitation and broad-spectrum ultraviolet light (UV-light) exposure.

The stability of Hu5F9-g4 in different formulations was examined under static storage conditions at freezing (−70° C.), refrigerated (5° C.) and accelerated temperatures (e.g. 25° C. and 37° C.) for up to 8 weeks. The majority of formulations remained clear and free of particles during temperature storage. However in the presence of an ionic tonicity modifier (sodium chloride) at pH 4.0, the Hu5F9-g4 turned hazy and displayed significant chemical and physical degradation after only 1 week at 37° C. WCX-HPLC analysis revealed chemical instability during elevated temperature storage for the product formulated at pH 7±1 and in the absence of an ionic tonicity modifier, especially at pH 6.0 and 6.5. The physical stability of Hu5F9-g4 was evaluated by SE-HPLC and SDS-PAGE. These examinations detected elevated levels of oligomeric or HMW species during accelerated temperature storage for the product formulated with a non-ionic tonicity modifier and in phosphate buffer, particularly at higher pH. In contrast, SE-HPLC analysis showed more HMW species in the presence of an ionic tonicity modifier during −70° C. storage.

A formulation at pH 5.0 with 10 mM acetate buffer, a non-ionic tonicity modifier (5% sorbitol) and polysorbate 20 (0.01%) was selected as the best formulation candidate from this study. This formulation demonstrated both chemical and physical stability during stress.

The stability results of the selected A5S formulation are summarized as follows. Most of the samples in this study remained clear and without particles after freeze/thaw, agitation, UV-light exposure and temperature stress. Thus, no visible physical degradation was observed for the formulation candidate ASS. The concentration for the selected formulation candidate remained on target over 8 weeks at −70° C., 5° C., 25° C. and 37° C. storage.

After 8 weeks of temperature storage, some Hu5F9-g4 formulations displayed a pH value slightly higher than target during the stability study. Nonetheless, the pH results for the selected candidate during temperature storage did not deviate more than ±0.2 pH units from T=0. The osmolality values for the formulation candidates were nearly isotonic. Samples formulated with sorbitol are slightly more hypertonic than those containing NaCl. The osmolality for the selected formulation candidate (A5S) was 314 mOsm.

Each of the 13 formulations that were tested generated comparable WCX-HPLC data following UV-light stress. No significant degradation was observed for the selected formulation candidate during this stress condition.

Compared to other formulations, the percent purity for A5S remained relatively high over 8 weeks of storage at −70° C., 5° C., 25° C. and 37° C.

SE-HPLC analysis revealed similar purity results for each sample after agitation, freeze/thaw and UV-light exposure. Thus, the selected formulation candidate displayed no considerable oligomerization or cleavage following these acute stress conditions.

SDS-PAGE examination exhibited no significant degradation for the selected candidate during storage at −70° C., 5° C., 25° C. and 37° C. The SDS-PAGE results for samples stored at both 25° C. and 37° C. for 8 weeks show that HMW species were only detected for the samples formulated at higher pH without NaCl.

TABLE 1
Components of initial formulation for Hu5F9-g4

| Ingredient | Concentration | | Purpose |
|---|---|---|---|
| Hu5F9-g4 | 10 | mg/mL | Active ingredient |
| Acetate | 10 | mM | Buffering agent |
| Sorbitol | 5% | (w/v) | Tonicity modifier |
| Polysorbate 20 (PS20) | 0.01% | (w/v) | Surfactant |

To the final pH of 5.0

### Example 2

### Evaluation of Final Formulation

Formulation Screening. Formulations with the following varying parameters were studied:

a. pH: 4 to 8

b. Buffer: 10 mM sodium acetate, 10 mM histidine, and 10 mM sodium phosphate

c. Stabilizer: 5% sorbitol (non-ionic) and 150 mM NaCl (ionic) The formulations evaluated are shown in Table 2.

TABLE 2
Formulation Compositions Evaluated

| Form. | Sample | | | Tonicity | [Hu5F9-g4] | |
|---|---|---|---|---|---|---|
| No. | ID | Buffer (10 mM) | pH | Modifier | (mg/mL) | Surfactant * |
| F1 | A4N | 10 mM Sodium Acetate | 4.0 | 150 mM NaCl | 10 | 0.01% PS20 |
| F2 | A4S | 10 mM Sodium Acetate | 4.0 | 5% Sorbitol | 10 | 0.01% PS20 |
| F3 | A5N | 10 mM Sodium Acetate | 5.0 | 150 mM NaCl | 10 | 0.01% PS20 |
| F4 | A5S | 10 mM Sodium Acetate | 5.0 | 5% Sorbitol | 10 | 0.01% PS20 |
| F5 | P6N | 10 mM Sodium Phosphate | 6.0 | 150 mM NaCl | 10 | 0.01% PS20 |
| F6 | P6S | 10 mM Sodium Phosphate | 6.0 | 5% Sorbitol | 10 | 0.01% PS20 |
| F7 | H6.5N | 10 mM Histidine | 6.5 | 150 mM NaCl | 10 | 0.01% PS20 |
| F8 | H6.5S | 10 mM Histidine | 6.5 | 5% Sorbitol | 10 | 0.01% PS20 |
| F9 | P7N | 10 mM Sodium Phosphate | 7.0 | 150 mM NaCl | 10 | 0.01% PS20 |
| F10 | P7S | 10 mM Sodium Phosphate | 7.0 | 5% Sorbitol | 10 | 0.01% PS20 |
| F11 | P8N | 10 mM Sodium Phosphate | 8.0 | 150 mM NaCl | 10 | 0.01% PS20 |
| F12 | P8S | 10 mM Sodium Phosphate | 8.0 | 5% Sorbitol | 10 | 0.01% PS20 |
| F13 | Control | PBS | 7.2 | NaCl | 10 | — |

* The surfactant chosen was based on agitation studies

The formulations were evaluated by the following stress conditions:

a. Thermal stability

b. Freeze/thaw (5 cycles from -70° C. to room temperature)

c. Agitation for 4 hours

d. Ambient light exposure for 24 hours

Surfactant Evaluation. Magrolimab (hu5F9-g4) at 11 mg/mL in PBS at pH 6.5 was spiked with 0.01% w/v polysorbate 20, 0.01% w/v polysorbate 80, and 0.10% F68 pluronic. These solutions were agitated for 4 hours at room temperature and evaluated for turbidity by visual appearance and absorbance at 600 nm (Table 2) and aggregate formation by SE-HPLC (Table 3). Turbidity measurements shows that PS20 and F68 resulted in less turbidity than PS80. However, overall, visual appearance results indicated that all 3 surfactants tested protected magrolimab from particle formation. For all 3 surfactants tested, there were no changes in the SE-HPLC quality profile.

TABLE 3
Turbidity Results

| Condition | Surfactant | Observations | A₆₀₀ |
|---|---|---|---|
| Agitated | none | cloudy (centrifuged) | 0.1767 |
| at room temp | 0.01% PS20 | clear solution | 0.0021 |
| for 4 hours | 0.01% PS80 | clear solution | 0.0169 |
| | 0.10% F-68 | clear solution | 0.0016 |

TABLE 4
SE-HPLC Results

| | | | | | Total |
|---|---|---|---|---|---|
| | | Pre-peak | Monomer | Post-peak | Area |
| Condition | Surfactant | % | % | % | (mAU) |
| Agitated | none | 2.5 | 97.5 | 0.0 | 28670 |
| at room temp | 0.01% PS20 | 2.5 | 97.4 | 0.1 | 28642 |
| for 4 hours | 0.01% PS80 | 2.5 | 97.4 | 0.1 | 28882 |
| | 0.10% F-68 | 2.5 | 97.4 | 0.1 | 28897 |
| Control | none (#1) | 2.5 | 97.4 | 0.1 | 28982 |
| no agitation | none (#2) | 2.6 | 97.3 | 0.1 | 28926 |

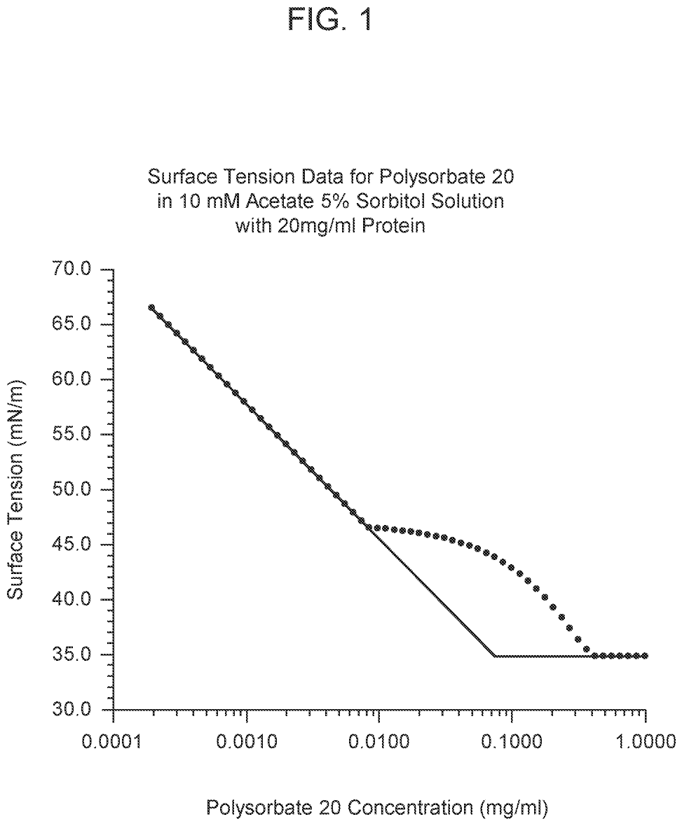

Surface tensiometry was performed to evaluate the interaction of polysorbate 20 to a formulation of 20 mg/mL magrolimab, 10 mM acetate, 5% w/v sorbitol, at pH 5.0. The surface tension profile is shown in FIG. 1. Polysorbate 20 starts interacting with the protein at approximately 0.01 mg/mL (0.001%). The protein in the solution is fully saturated with polysorbate 20 at approximately 0.1-0.2 mg/mL (0.01% to 0.02%).

Formulation 1 containing 10 mM sodium acetate, 150 mM NaCl at pH 4.0 resulted in rapid degradation. This formulation was not further evaluated in any stressed condition study.

The results of the freeze/thaw, agitation, and ambient light exposure studies are summarized in the table below.

TABLE 5

| Stress | | |
|---|---|---|
| Condition | Analytical Methods | Results |
| Freeze/thaw | Visual Appearance | All formulations F2 through F13 |
| | SE-HPLC | were clear and free of particulates. |
| | | All formulations had acceptable |
| | | SE-HPLC profiles after 5 |
| | | freeze/thaw cycles. However, |
| | | formulations containing NaCl |
| | | resulted in slightly higher |
| | | aggregate formation. |
| Agitation | Visual Appearance | All formulations F2 through F13 |
| | SE-HPLC | were clear and free of particulates |
| | | and no changes in SE-HPLC profile. |
| Ambient | Visual Appearance | All formulations F2 through F13 |
| light | SE-HPLC | were clear and free of particulates |
| exposure | WCX-HPLC | and stable to ambient light exposure |
| | SDS-PAGE | with no major changes in SE-HPLC |
| | (reduced | and WCX-HPLC profiles and |
| | and non-reduced) | SDS-PAGE results. |

Thermal Stability. The formulations were placed on stability for 8 weeks at −70° C., 5° C., 25° C., and 37° C. The formulations were evaluated by protein concentration, pH, visual appearance, SE-HPLC, WCX-HPLC, and SDS-PAGE reduced and non-reduced.

After 8 weeks, all formulations except for formulation 1 was clear and particle-free at all temperatures tested. Formulation 1 was hazy after 1 week at 37° C. and was discontinued from the study.

The protein concentration remained unchanged after 8 weeks as shown in Table 6.

TABLE 6
Protein Concentration results

| | | Concentration (mg/mL) results | | | | |
|---|---|---|---|---|---|---|
| Form. | Sample | | T = 8 weeks stress | | | |
| No. | ID | T = 0 | −70° C. | 4° C. | 25° C. | 37° C. |
| F1* | A4N | 9.8 | NA | NA | NA | NA |
| F2 | A4S | 10.2 | 10.2 | 10.2 | 10.2 | 10.3 |
| F3 | A5N | 9.8 | 10.0 | 10.2 | 10.2 | 10.2 |
| F4 | A5S | 9.9 | 10.1 | 9.9 | 9.9 | 10.1 |
| F5 | P6N | 9.9 | 10.4 | 10.3 | 10.3 | 10.3 |
| F6 | P6S | 10.0 | 10.0 | 10.3 | 10.3 | 10.3 |
| F7 | H6.5N | 10.1 | 10.4 | 10.5 | 10.5 | 10.5 |
| F8 | H6.5S | 9.8 | 9.9 | 10.2 | 10.3 | 10.3 |
| F9 | P7N | 10.0 | 10.2 | 10.3 | 10.3 | 10.3 |
| F10 | P7S | 9.9 | 10.3 | 10.2 | 10.3 | 10.3 |
| F11 | P8N | 10.0 | 10.0 | 10.2 | 10.2 | 9.9 |
| F12 | P8S | 10.0 | 9.8 | 10.1 | 10.0 | 9.9 |
| F13 | Control | 9.9 | 9.4 | 9.4 | 9.4 | 9.4 |

*This sample was no longer analyzed following the 1 week time point

The pH was measured at the end of the study. Generally the pH was higher than the initial time point but within the error of the measurement.

TABLE 7
pH results

| | | | pH results | | | | |
|---|---|---|---|---|---|---|---|
| Form. | Sample | Target | | T = 8 weeks stress | | | |
| No. | ID | pH | T = 0 | −70° C. | 4° C. | 25° C. | 37° C. |
| F1* | A4N | 4.0 | 4.0 | NA | NA | NA | NA |
| F2 | A4S | 4.0 | 4.1 | 4.4 | 4.4 | 4.4 | 4.5 |
| F3 | A5N | 5.0 | 5.0 | 5.2 | 5.2 | 5.3 | 5.3 |
| F4 | A5S | 5.0 | 5.1 | 5.2 | 5.2 | 5.3 | 5.3 |
| F5 | P6N | 6.0 | 5.9 | 6.2 | 6.3 | 6.3 | 6.3 |
| F6 | P6S | 6.0 | 6.1 | 6.3 | 6.4 | 6.4 | 6.4 |
| F7 | H6.5N | 6.5 | 6.5 | 6.6 | 7.2 | 7.9 | 6.5 |
| F8 | H6.5S | 6.5 | 6.6 | 6.7 | 6.8 | 6.2 | 6.2 |
| F9 | P7N | 7.0 | 7.0 | 7.3 | 7.3 | 7.3 | 7.2 |
| F10 | P7S | 7.0 | 7.1 | 7.3 | 7.3 | 7.3 | 7.3 |
| F11 | P8N | 8.0 | 7.9 | 7.8 | 7.8 | 7.8 | 7.9 |
| F12 | P8S | 8.0 | 7.9 | 7.9 | 7.9 | 7.9 | 7.8 |
| F13 | Control | 7.2 | 7.2 | 7.5 | 7.6 | 7.6 | 7.5 |

*This sample was no longer analyzed following the 1 week time point

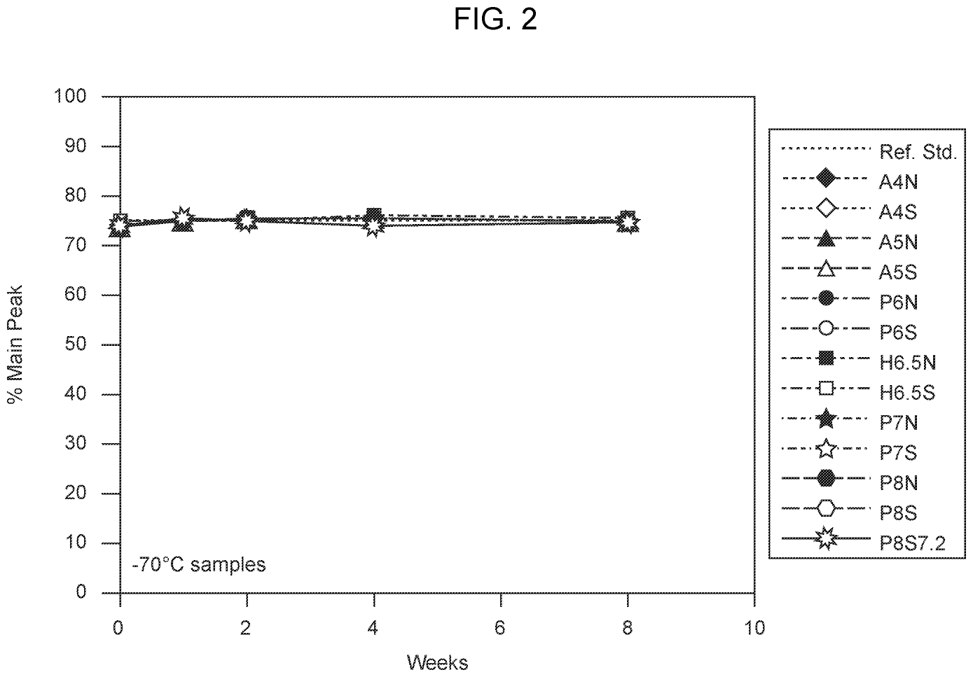

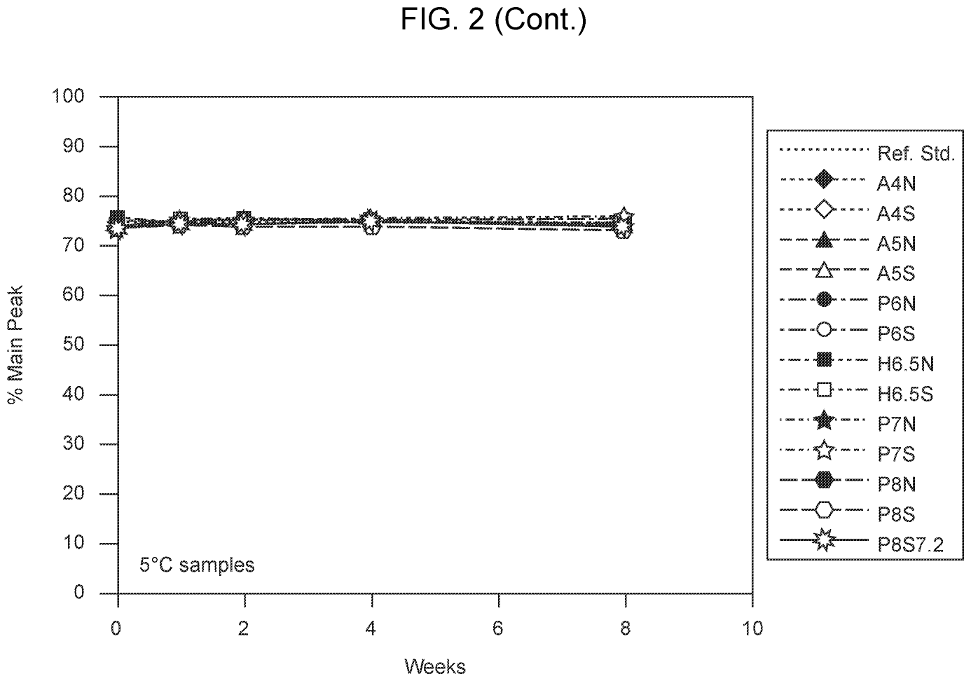

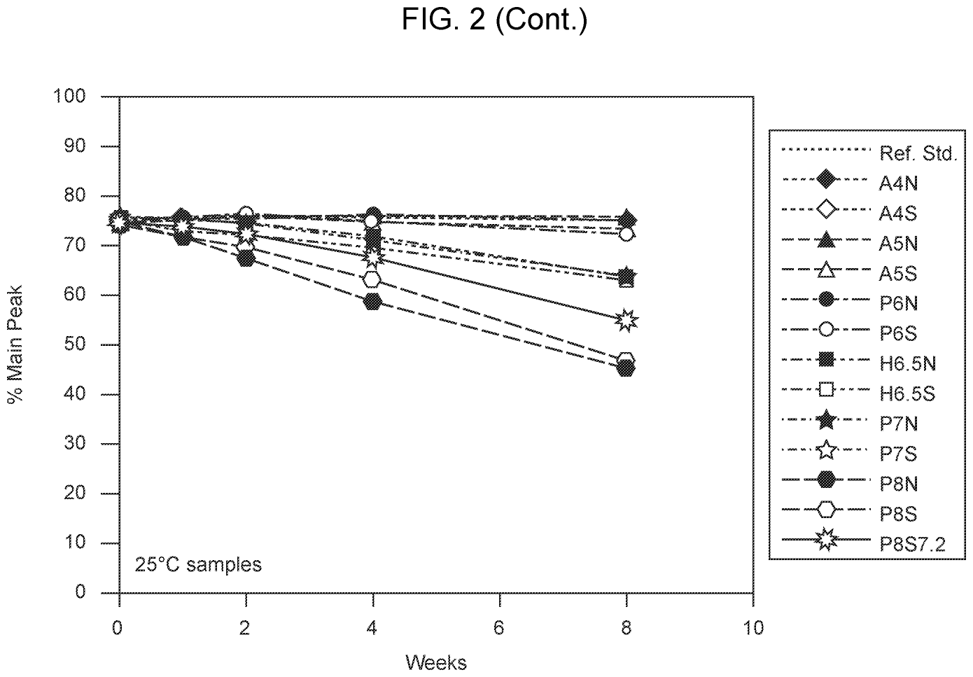

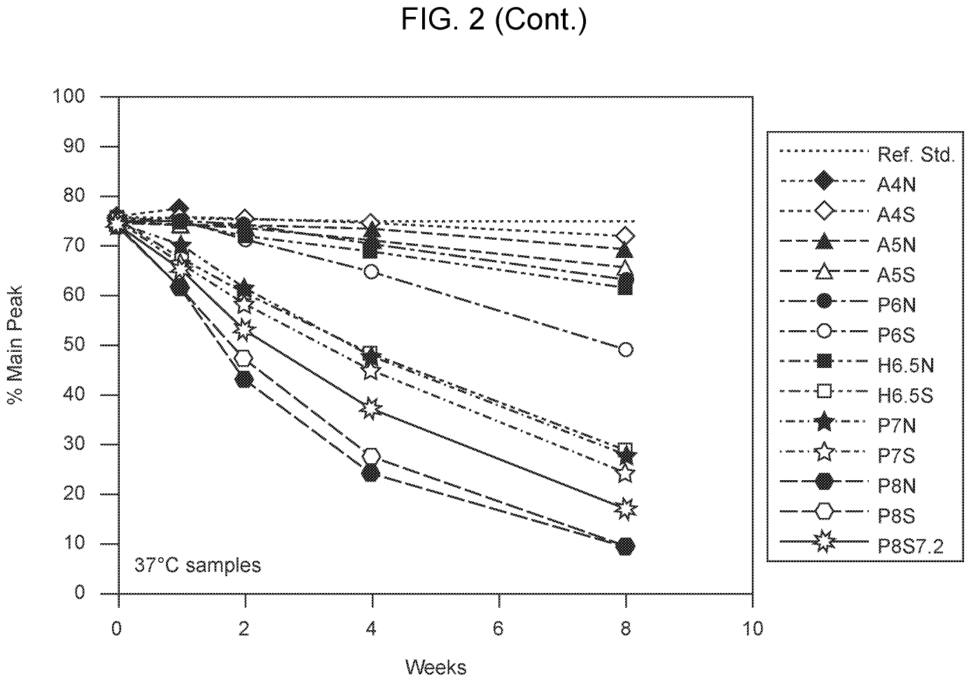

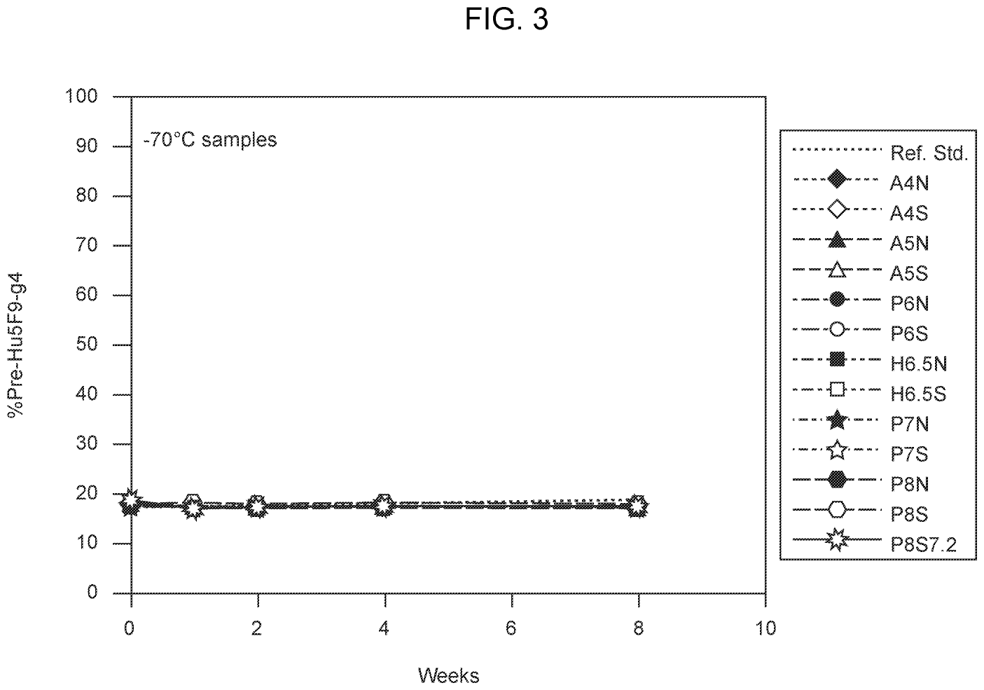

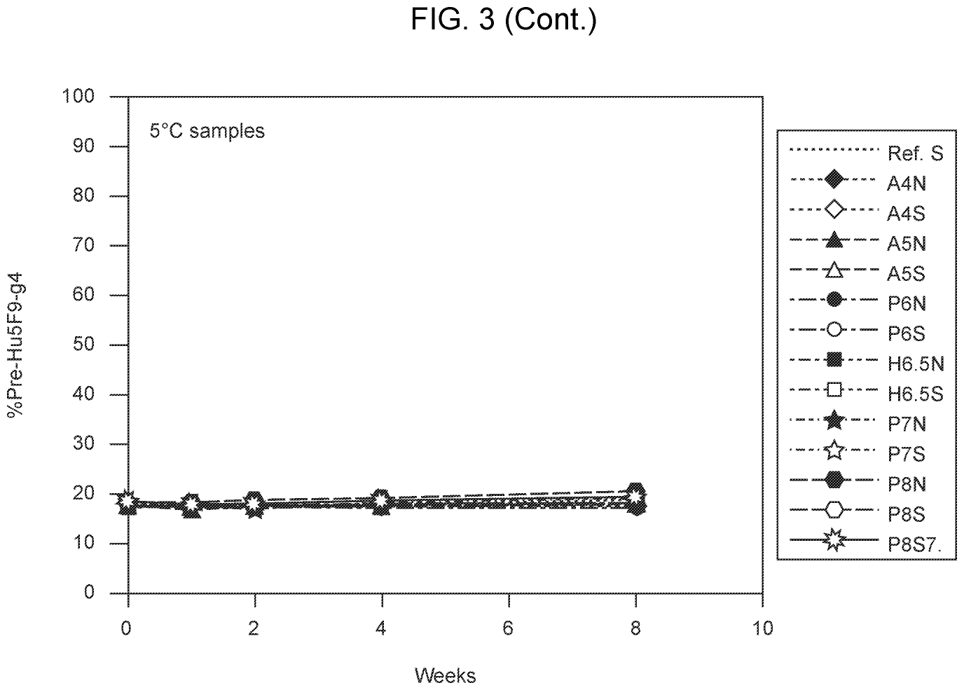

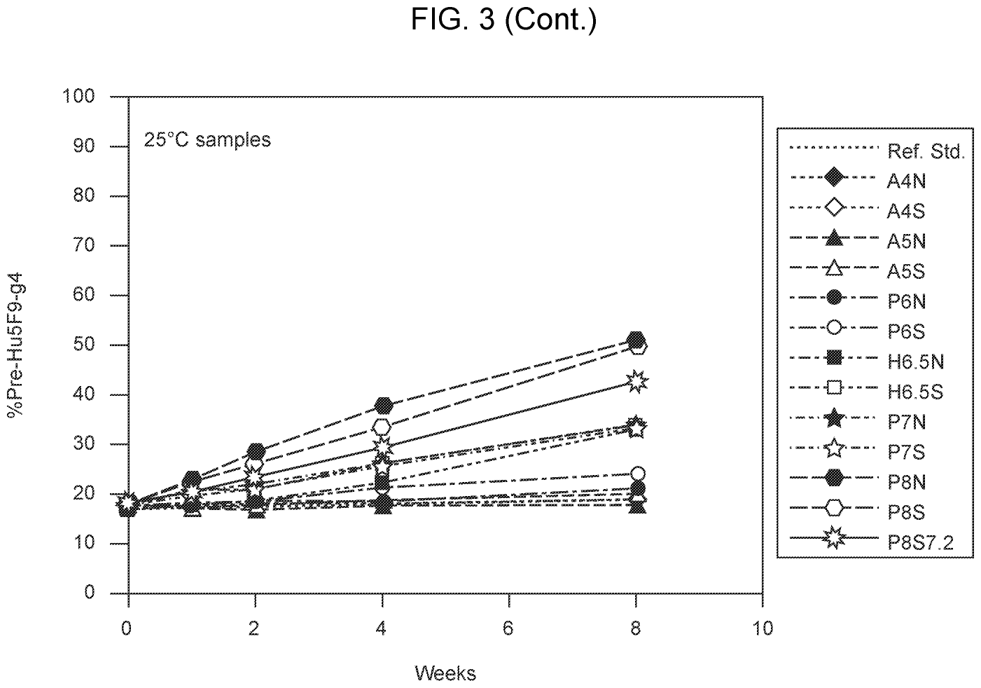

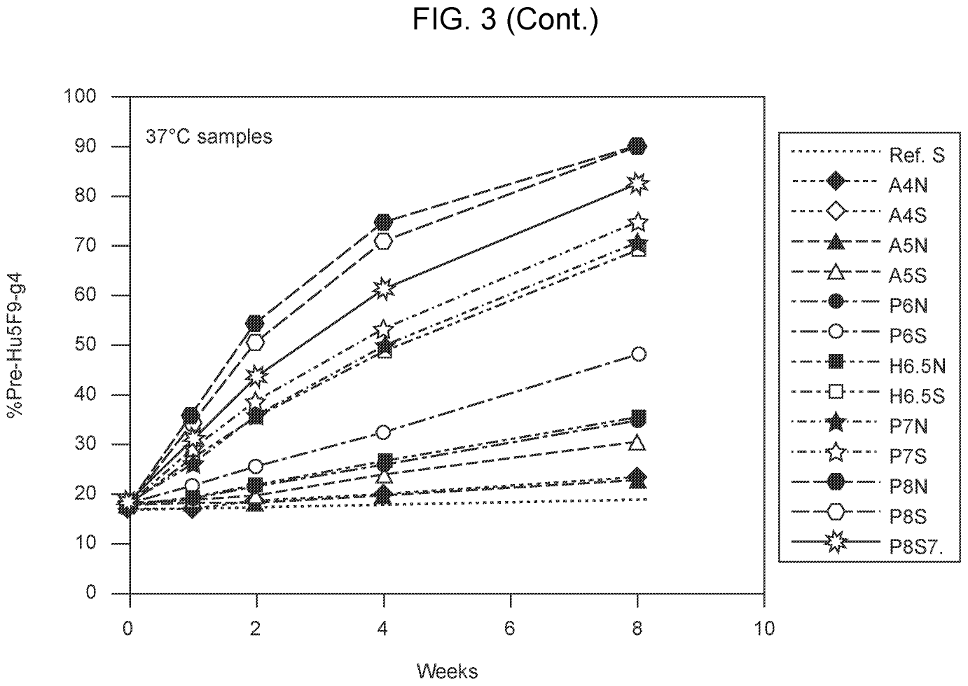

The WCX-HPLC main peak and pre-peak profiles of all the formulations at the various storage temperatures up to 8 weeks storage were determined, shown in FIGS. 2 and 3. At −70° C. and 5° C. storage, there is no change up to 8 weeks. At 25° C. and 37° C., the formulations with the least amount of decrease in the main peak are formulations at pH 5 and pH 6. At pH 4, the sorbitol formulation is more stable than the NaCl formulation, while at pH 6.5 the sorbitol formulation is less stable than the NaCl formulation. As the main peak decreases, the pre-peak/acidic species increases, with similar stability trends in pH and stabilizer.

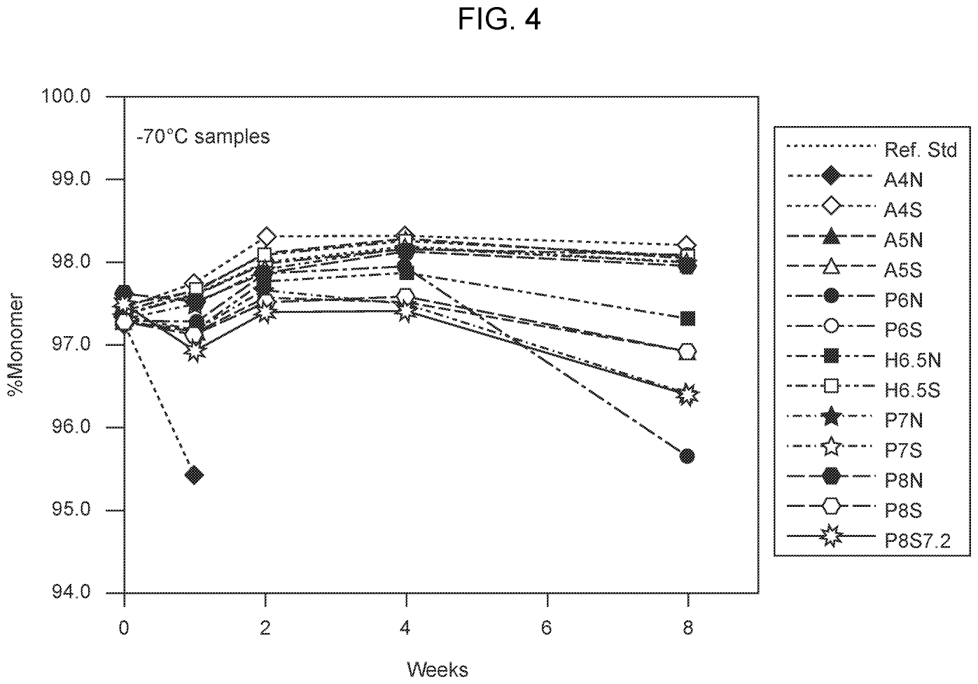

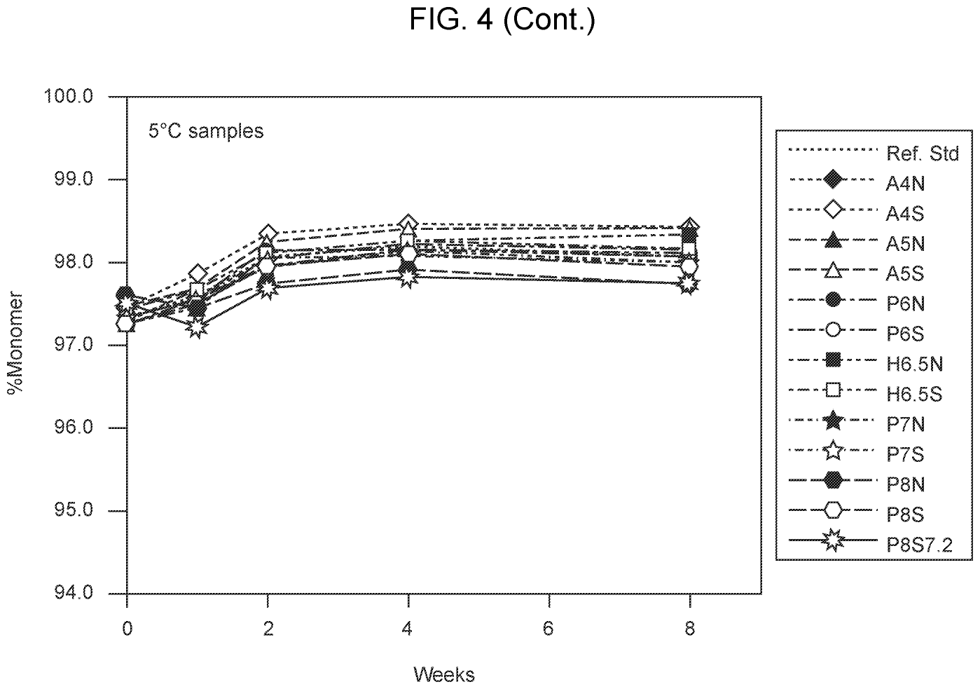

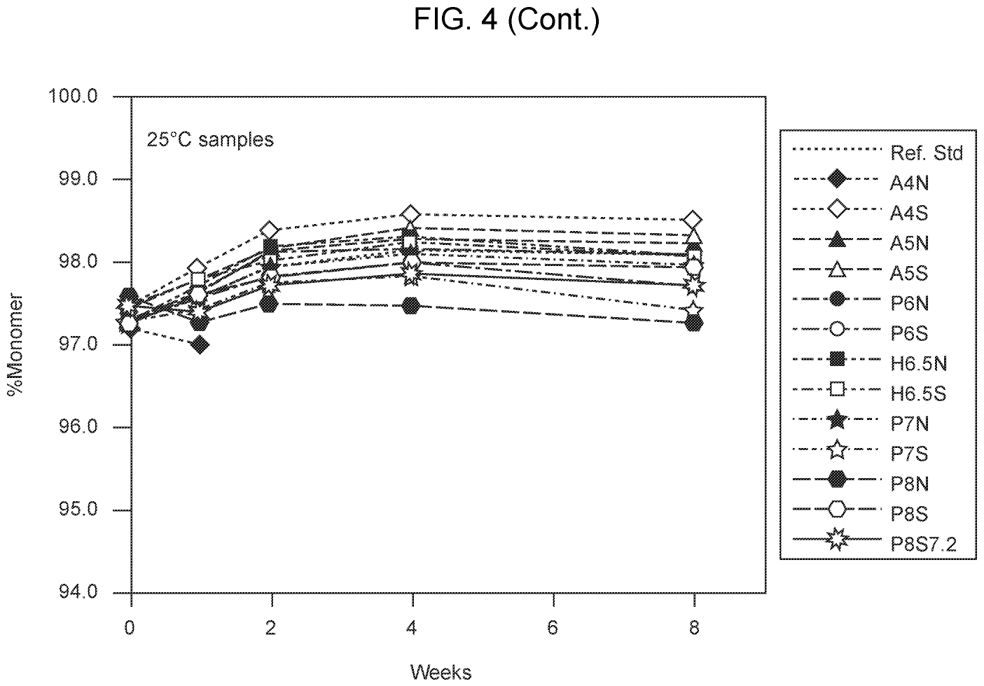

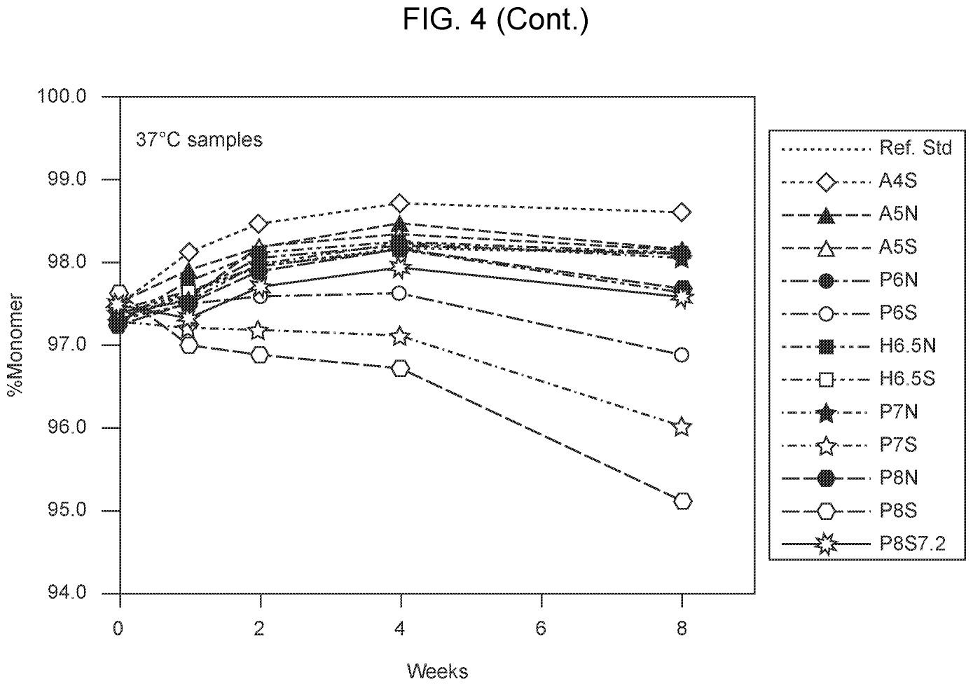

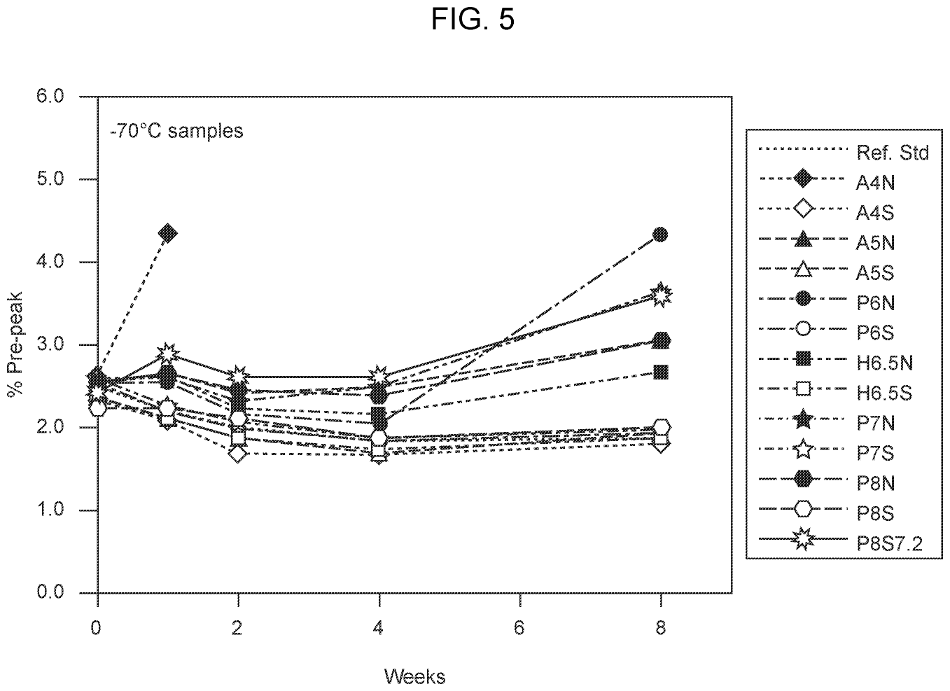

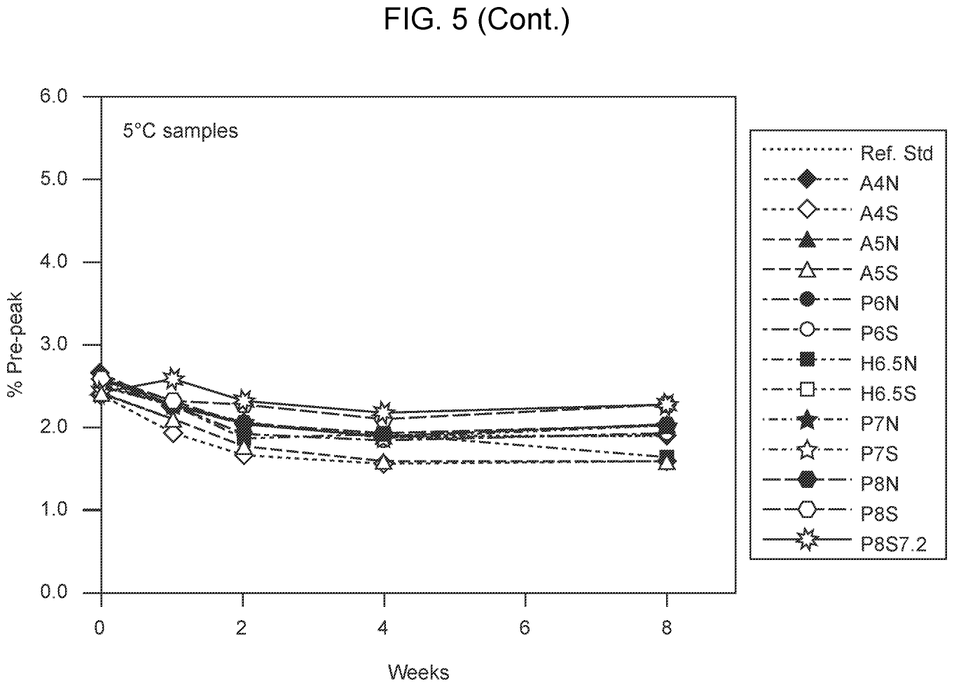

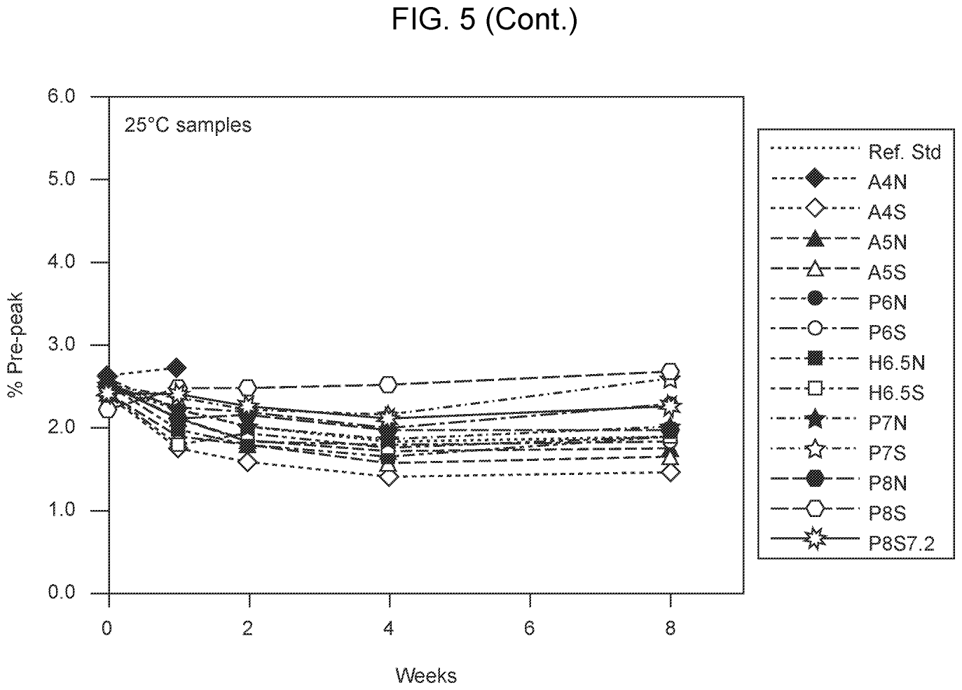

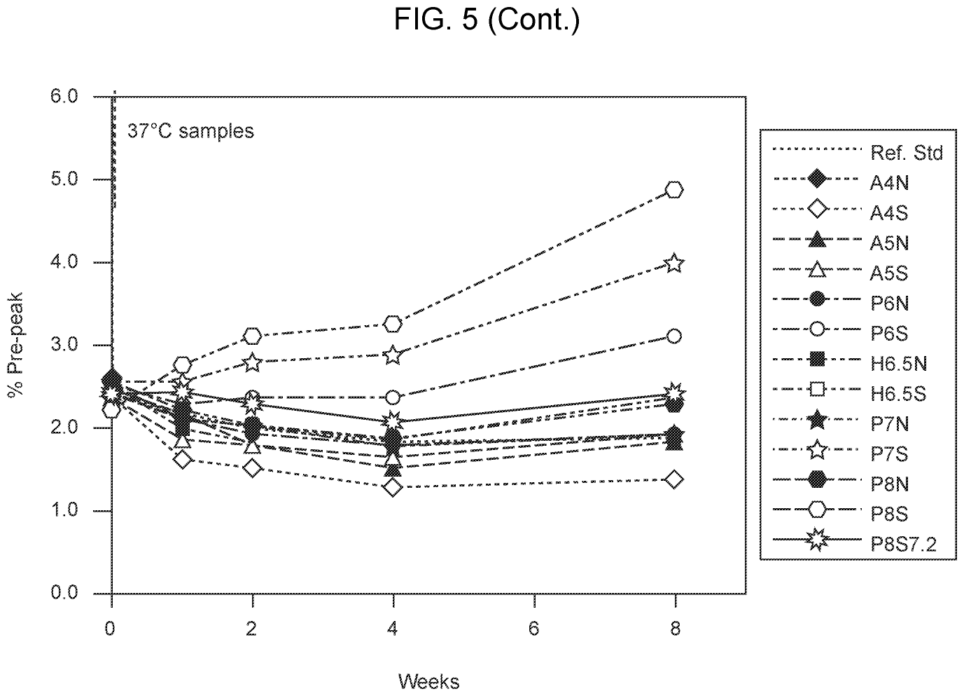

The SE-HPLC monomer and aggregates/pre-peak profiles of all the formulations at the various storage temperatures up to 8 weeks storage are shown in FIG. 4 and FIG. 5, respectively. At 5° C. and 25° C. storage, there is no significant change up to 8 weeks. At -70° C. and at 37° C., there is a decrease in monomer, with concomitant increase in aggregates. At -70° C., formulations from pH 6 to 8 containing NaCl or PBS resulted in higher aggregate formation. At 37° C., formulations containing phosphate buffer and sorbitol from pH 6-8 had higher rates of aggregate formation.

The results for the stable formulation parameter space is summarized in Table 8.

TABLE 8
Stable Formulation Excipient Parameter Space

| Formulation | pH Stable | |
|---|---|---|
| Excipient | Range | Attributes Evaluated |
| NaCl | 5.0-6.5 | WCX-HPLC |
| | 5.0 | SE-HPLC (at −70° C., frozen |
| | | liquid formulation) |
| | 5.0-8.0 | SE-HPLC (at 37° C., as liquid |
| | | formulation) |
| Sorbitol | 4.0-6.0 | WCX-HPLC |
| | 4.0-6.5 | SE-HPLC |

Long Term Stability of Selected Formulation. Based on the development studies, the formulation selected for a long-term study was 20 mg/mL magrolimab, 10 mM acetate, 5% w/v sorbitol, 0.01% polysorbate 20 at pH 5.0. This formulation is stored in Type 1 glass vials with fluropolymer coated butyl stoppers, and it is stable at 5° C. for at least 3 years as measured by SE-HPLC, icIEF, CE-SDS reduced and non-reduced, and binding, as detailed in the table below.

TABLE 9
Stability of 20 mg/mL magrolimab, 10 mM acetate, 5% w/v sorbitol, 0.01% polysorbate
20 at pH 5.0 Stored at 5° C.

| | | | | | | | CE-SDS | | | |
|---|---|---|---|---|---|---|---|---|---|---|
| | | | | | | CE-SDS | Non- | | | |
| Time | | Protein | icIEF | icIEF | icIEF | Reduced | reduced | SEC | SEC | |
| Point | | Concentration | % | % | % | (HC + LC) | Main | Monomer | Aggregates | Binding |
| (months) | Particles | (mg/mL) | Acidic | main | basic | % | (%) | (%) | (%) | (%) |
| 0 | FVP | 21 | 37.9 | 56.3 | 5.8 | 98 | 97.4 | 98.6 | 1.3 | 84.9 |
| 1 | FVP | 21.2 | 38.0 | 55.8 | 6.2 | 97.8 | 97.1 | 98.4 | 1.5 | 88.9 |
| 3 | FVP | 20.4 | 37.1 | 56.1 | 6.8 | 97.6 | 97.7 | 98.6 | 1.3 | 81.1 |
| 6 | FVP | 20.7 | 37.1 | 56.2 | 6.8 | 98.1 | 97.2 | 98.6 | 1.3 | 95.9 |
| 9 | FVP | 20.6 | 38.6 | 55.1 | 6.3 | 98.3 | 97 | 98.6 | 1.3 | 100.1 |
| 12 | FVP | 21.4 | 37.6 | 56.5 | 5.9 | 98.5 | 97.2 | 98.6 | 1.4 | 109 |
| 24 | FVP | 20.8 | 37.3 | 55.6 | 7.7 | 98.4 | 97.2 | 98.5 | 1.4 | 97 |
| 36 | FVP | 20.8 | 38.5 | 54.5 | 7.1 | 98.2 | 97.3 | 98.50 | 1.5 | 102 |

FVP = free from visible particles

### Example 3

### Viscosity Measurement of Magrolimab

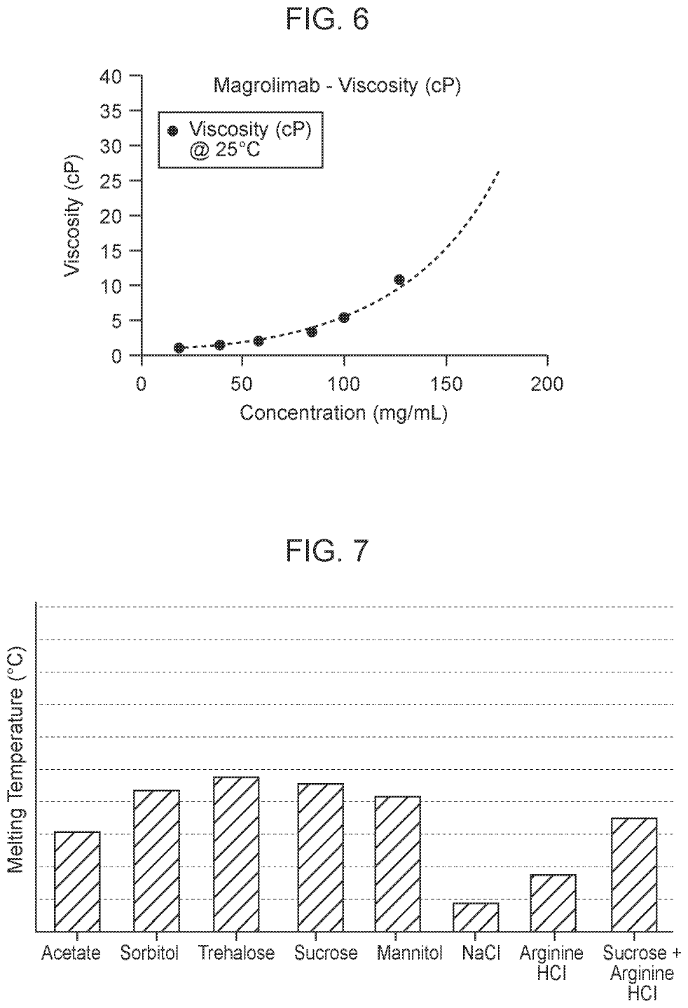

The viscosity of magrolimab was measured as a function of concentration. The protein was concentrated to more than 120 mg/mL still remaining in solution and was visually clear. The protein viscosity is low, reaching approximately 20 cP at 160 mg/mL. The results are depicted in FIG. 6.

### Example 4

### Turbidity Evaluation of Magrolimab Formulations

Shaking studies were performed with magrolimab with different concentrations of polysorbate 20. With zero polysorbate 20 in the formulation, the solution exhibited increased turbidity and formation of aggregates measured by SEC-HPLC, and subvisible particles. With 0.005% and higher concentrations of polysorbate 20, there was no change in the formulation quality attributes. This result correlated well with the surface tensiometry data.

| | | | Subvisible | |
|---|---|---|---|---|
| Polysorbate 20 | | | Particles | |
| Concentration | Turbidity | SEC-HPLC % | ≥10 μm | ≥25 μm |
| (% w/v) | (NTU) | HMW | particles/mL | particles/mL |
| 0.0 | 30 | 8.3 | 690461 | 5632 |
| 0.005 | 6 | 1.8 | 118 | 0 |
| 0.010 | 6 | 1.6 | 171 | 0 |
| 0.015 | 6 | 1.6 | 91 | 0 |

### Example 5

### Excipient Screen with Magrolimab

Various stabilizing excipients were studied with magrolimab and the melting temperature was evaluated by intrinsic fluorescence. The melting temperature and stability data shown in FIG. 7 indicate that all excipients tested stabilize magrolimab. The melting temperatures indicated that magrolimab was most stabilized in the presence of the saccharide excipients.

What is claimed is:

1. A pharmaceutical stable aqueous formulation comprising 10-100 mg/ml of an anti-CD47 antibody;
and pharmaceutically acceptable excipients comprising a buffer at a concentration of 5-20 mM; at least one stabilizer, and a surfactant, wherein the formulation has a pH of from about pH 4-6.5.

2. The formulation of claim 1, wherein the buffer is present at a concentration of from 10 to 15 mM.

3. The formulation of any of claims 1-2, wherein the buffer in the formulation is acetate buffer or histidine buffer.

4. The formulation of claim 3, wherein the buffer is acetate buffer.

5. The formulation of any of claims 1-4, where the pH is from pH 4 to pH 6.5.

6. The formulation of claim 5, where the pH is from pH 4.7 to pH 5.3.

7. The formulation of claim 5, where the pH is from pH 4.9 to pH 5.1.

8. The formulation according to any of claims 1-7, wherein the stabilizer is sorbitol at a concentration of from 2.5% to 10%.

9. The formulation according to any of claims 1-7, wherein the stabilizer is sucrose at a concentration of from 2.5% to 10%.

10. The formulation according to any of claims 1-7, wherein the stabilizer is trehalose at a concentration of from 2% to 12%.

11. The formulation according to any of claims 1-7, wherein the stabilizer is NaCl at a concentration of from 100-200 mM.

12. The formulation according to any of claims 1-11, wherein the surfactant is polysorbate 20, polysorbate 80, or pluronic F68.

13. The formulation of claim 12, wherein the surfactant is polysorbate 20 at a concentration of surfactant of from about 0.005% to about 0.05%.

14. The formulation of any of claims 1-13, wherein the formulation is stable at a temperature of from about 2° C. to about 8° C. for at least 12 months.

15. The formulation of any of claims 1-13, wherein the formulation is stable at a temperature of from about 2° C. to about 8° C. for at least 16 weeks.

16. The formulation of claim 15, wherein the anti-CD47 antibody in the formulation retains at least 50% of its biological activity after storage.

17. The formulation of any of claims 1-16, wherein the anti-CD47 antibody in the formulation is a full-length antibody.

18. The formulation of claim 17, wherein the antibody comprises a human IgG4 constant region sequence.

19. The formulation of claim 17 or 18, wherein the antibody is a humanized antibody comprising an HCDR1 region comprising of sequence (SEQ ID NO: 1), an HCDR2 region comprising of sequence (SEQ ID NO: 2), an HCDR3 region comprising of sequence (SEQ ID NO: 3, an LCDR1 region comprising of sequence (SEQ ID NO: 4), an LCDR2 region comprising of sequence (SEQ ID NO: 5), and an LCDR3 region comprising of sequence (SEQ ID NO: 6).

20. The formulation according to any of claims 1-19, wherein the antibody is magrolimab.

21. A pharmaceutically acceptable, stable liquid formulation comprising 10-20 mg/ml magrolimab; 10 mM acetate buffer; 5% w/v sorbitol; 0.01%-0.04% polysorbate 20 at a pH of from 4.5-5.5.

22. A pharmaceutically acceptable, stable liquid formulation comprising 10-20 mg/ml magrolimab; 10 mM acetate buffer; 9% w/v sucrose; 0.01%-0.04% polysorbate 20 at a pH of from 4.5-5.5.

23. A pharmaceutically acceptable, stable liquid formulation comprising 10-20 mg/ml magrolimab; 10 mM acetate buffer; 9% w/v trehalose; 0.01%-0.04% polysorbate 20 at a pH of from 4.5-5.5.

24. An article of manufacture comprising a unit dose of a formulation according to any of claims 1-23.

25. A method of treating an individual in need thereof by administering an effective dose of an antibody formulation according to any of claims 1-23.


SEQUENCE LISTING (12 sequences)

SEQ ID NO: 1 - protein, 5 aa, Mus musculus
NYNMH

SEQ ID NO: 2 - protein, 17 aa, Mus musculus
TIYPGNDDTSYNQKFKD

SEQ ID NO: 3 - protein, 8 aa, Mus musculus
GGYRAMDY

SEQ ID NO: 4 - protein, 16 aa, Mus musculus
RSSQSIVYSNGNTYLG

SEQ ID NO: 5 - protein, 7 aa, Mus musculus
KVSNRFS

SEQ ID NO: 6 - protein, 9 aa, Mus musculus
FQGSHVPYT

SEQ ID NO: 7 - protein, 117 aa, Artificial Sequence
  Humanized antibody hu5F9-vh1
QVQLVQSGAEVKKPGASVKVSCKASGYTFTNYNMHWVRQAPGQGLEWIGTIYPGNDDTSYNQKFKDKATLTADKSTSTAYMELSSLRSEDTAVYYCARGGYRAMDYWGQGTLVTVSS

SEQ ID NO: 8 - protein, 117 aa, Artificial Sequence
  Humanized antibody hu5F9-vh2
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 9 - protein, 117 aa, Artificial Sequence
  Humanized antibody hu5F9-vh3
QVQLVQSGAEVKKPGASVKVSCKASGYTFTNYNMHWVRQAPGQRLEWIGTIYPGNDDTSYNQKFKDRATLTADKSASTAYMELSSLRSEDTAVYYCARGGYRAMDYWGQGTLVTVSS

SEQ ID NO: 10 - protein, 112 aa, Artificial Sequence
  Humanized antibody hu5F9-vl1
DVVMTQSPLSLPVTPGEPASISCRSSQSIVYSNGNTYLGWYLQKPGQSPKLLIYKVSNRFSGVPDRFSGSGSGTDFTLKISRVEAEDVGVYHCFQGSHVPYTFGGGTKVEIK

SEQ ID NO: 11 - protein, 112 aa, Artificial Sequence
  Humanized antibody hu5F9-vl2
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 12 - protein, 112 aa, Artificial Sequence
  Humanized antibody hu5F9-vl3
DVVMTQSPLSLPVTPGEPASISCRSSQSIVYSNGNTYLGWYLQKPGQSPQLLIYKVSNRFSGVPDRFSGSGSGTDFTLKISRVEAEDVGVYHCFQGSHVPYTFGQGTKLEIK